(19) United States
(12) Patent Application Publication
(10) Pub. No.: US 2024/0295570 A1
(43) Pub. Date: Sep. 5, 2024

(54) METHOD FOR IDENTIFYING CANDIDATE THERAPEUTICS FOR ALZHEIMER'S DISEASE

(71) Applicant: UNIVERSITY OF SOUTH FLORIDA, Tampa, FL (US)

(72) Inventor: Gopal Thinakaran, Tampa, FL (US)

(21) Appl. No.: 18/594,431

(22) Filed: Mar. 4, 2024

Related U.S. Application Data
(60) Provisional application No. 63/487,962, filed on Mar. 2, 2023.

(51) Int. Cl. G01N 33/68; C12P 21/02

(52) U.S. Cl. CPC G01N 33/6896; C12P 21/02

(57) ABSTRACT

Provided herein are isolated BIN1 SH3 domain proteins and methods for their use to identify candidate Alzheimer's disease therapeutics.

### CROSS-REFERENCE TO RELATED APPLICATIONS

This patent application claims the benefit of, and priority to, U.S. Provisional Patent Application No. 63/487,962, filed Mar. 2, 2023, the content of which is incorporated herein by reference in its entirety.

### REFERENCE TO AN ELECTRONIC SEQUENCE LISTING

The contents of the electronic sequence listing (173738.02738.xml; Size: 2,839 bytes; and Date of Creation: Mar. 2, 2024) is herein incorporated by reference in its entirety.

### BACKGROUND OF THE INVENTION

Alzheimer's disease (AD) is a neurodegenerative condition characterized by cognitive deficits through accumulation of extracellular amyloid plaques and intracellular neurofibrillary tangles that eventually result in synaptic loss and neuronal cell death. Bridging Integrator 1 (BIN1) has been identified as a significant genetic risk factor locus for late-onset Alzheimer's disease (LOAD) identified by genome-wide association studies. Therefore, BIN1 is a promising therapeutic target for treating AD. Identifying therapeutics that can inhibit BIN1 are of interest.

### SUMMARY OF THE INVENTION

In an aspect, provided herein is an isolated protein comprising a sequence having at least 95% identity to SEQ ID NO: 1. In embodiments, isolated protein comprises the sequence of SEQ ID NO: 1. In embodiments, the isolated protein consists of the sequence of SEQ ID NO: 1.

In embodiments, the isolated protein is linked to a solid support.

In another aspect, provided herein is a polynucleotide comprising a sequence encoding the isolated protein of claim 1. In embodiments, the polynucleotide further comprises a sequence encoding a purification tag. In embodiments, the purification tag is a His tag. In embodiments, the sequence encoding the isolated protein and the His tag has at least 95% identity to SEQ ID NO: 2.

In another aspect, provided herein is an expression vector comprising the polynucleotide encoding any of the isolated proteins described herein. In embodiments, the expression vector is a bacterial vector.

In another aspect, provided herein is a host cell comprising any of the expression vectors described herein. In embodiments, the host cell is a bacterial cell.

In another aspect, provided herein is kit comprising any of the polynucleotides described herein, and a cell-free protein synthesis system.

In another aspect, provided herein is a method for identifying a candidate therapeutic for treating Alzheimer's disease, the method comprising: determining an equilibrium dissociation constant (KD) between an analyte and an isolated protein, wherein the isolated protein comprises a sequence having at least 95% identity to SEQ ID NO: 1; wherein a KD of less than about 15 μM identifies the analyte as a candidate therapeutic.

In embodiments, a KD of between about 0.1 μM and about 15 μM identifies the analyte as a candidate therapeutic. In embodiments, the step of determining the KD is done by surface plasmon resonance. In embodiments, the analyte is a chemical compound.

### BRIEF DESCRIPTION OF THE DRAWINGS

FIG. 1 is a schematic of BIN1 exons and alternate splicing. The C-terminal SH3 domain is present in all BIN1 isoforms.

FIG. 2 shows BIN1-SH3 domain crystals grown in 0.1 M MES (pH 6.5), 40% PEG 400, 5% PEG 3350.

FIG. 3 is the crystal structure of the human BIN1-SH3 domain, chain A.

FIG. 4 illustrates docking of a lead-like compound to BIN1's SH3 domain.

FIG. 5 shows SPR on FL 0N4R WT Tau and native BIN1 SH3 domain.

FIG. 6 shows SPR of nine candidate BIN1-SH3-binding compounds.

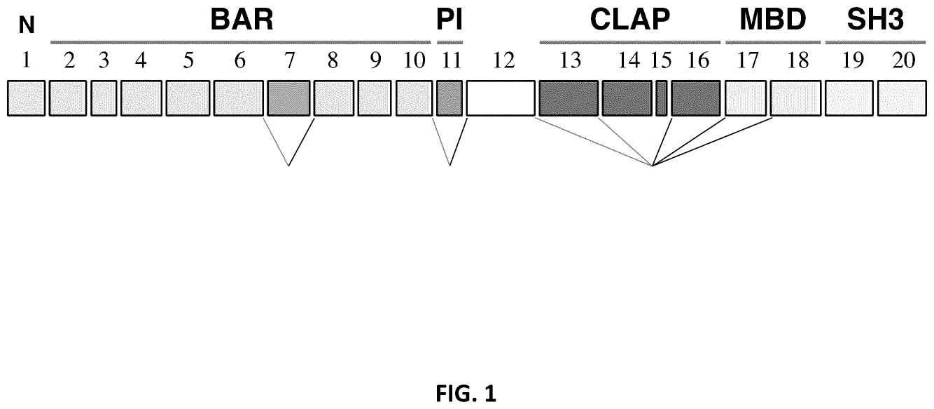

### DETAILED DESCRIPTION

Genome-wide association studies have identified Bridging Integrator 1 (BIN1) as a leading susceptibility locus for the development of late-onset AD. AD-associated BIN1 variants are thought to increase its expression. BIN1 is alternatively spliced to form various tissue/cell-type-specific, and ubiquitous, isoforms that engage in multiple cellular pathways including endocytosis, cytoskeletal remodeling, generating membrane curvature, and synaptic transmission. All BIN1 isoforms contain a c-terminal SH3 domain, an evolutionarily conserved adaptor module that facilitates transient protein-protein interactions typically though hydrophobic interactions among side chains. Therefore, this domain is a promising therapeutic target for treating Alzheimer's disease. As described herein, the inventors have isolated the BIN1 SH3 domain and used it to identify small molecule inhibitors for use in future therapeutic studies.

In a first aspect, provided herein is an isolated protein (BIN1 SH3 domain) comprising SEQ ID NO: 1 (GRLDLPPGFMFKVQAQHDYTATDTDELQLKAGDVVLVIPFQNPEEQDEGWLMGVKE SDWNQHKELEKCRGVFPENFTERVP), or a sequence having at least 80%, 85%, 90%, 91%, 92%, 93%, 94%, 95%, 96%, 97%, 98%, or 99% identity thereto. The isolated protein may consist of SEQ ID NO: 1.

As used herein, the terms “protein”, “polypeptide”, and “peptide” are used interchangeable to refer to a polymer of amino acids. Typically, a “polypeptide” or “protein” is defined as a longer polymer of amino acids, of a length typically of greater than 50, 60, 70, 80, 90, or 100 amino acids. A “peptide” is defined as a short polymer of amino acids, of a length typically of 50, 40, 30, 20 or less amino acids.

The proteins disclosed herein include wild type proteins and variants, mutants, and derivatives thereof. As used herein the term “wild type” is a term of the art understood by skilled persons and means the typical form of an organism, strain, gene or characteristic as it occurs in nature as distinguished from mutant or variant forms. As used herein, a “variant, “mutant,” or “derivative” refers to a protein molecule having an amino acid sequence that differs from a reference protein or polypeptide molecule. A variant or mutant may have one or more insertions, deletions, or substitutions of an amino acid residue relative to a reference molecule. A variant or mutant may include a fragment of a reference molecule. For example, a mutant or variant molecule may one or more insertions, deletions, or substitution of at least one amino acid residue relative to a reference polypeptide.

An “isolated” or “purified” protein or polynucleotide is removed from its natural environment, such as a cell, and is at least 60% free, preferably at least 75% free, and more preferably at least 90% free, even more preferably at least 95% free from other components with which it is naturally associated.

“Substantial identity” of amino acid sequences means that a polypeptide comprises a sequence that has at least 85% sequence identity to a reference sequence (SEQ ID NO) using a sequence alignment program; preferably BLAST using standard parameters. A preferred percent identity of polypeptides can be any integer from 85% to 100%. A preferred percent identity may be 85%, 86%, 87%, 88%, 89%, 90%, 91%, 92%, 93%, 94%, 95%, 96%, 97%, 98%, or 99% to a reference sequence.

In a second aspect, provided herein is a polynucleotide comprising a sequence encoding the isolated BIN1 SH3 protein. The polynucleotide may be included in a construct for expressing the isolated protein.

The terms “polynucleotide,” “polynucleotide sequence,” “nucleic acid” and “nucleic acid sequence” refer to deoxyribonucleic acid (DNA), ribonucleic acid (RNA) and DNA/RNA hybrids. Polynucleotides may be single-stranded or double-stranded. Nucleic acids include, but are not limited to: genomic DNA (gDNA), complementary DNA (cDNA), synthetic RNA, synthetic DNA, or recombinant DNA.

As used herein, the term “construct” refers to a recombinant polynucleotide, i.e., a polynucleotide that was formed artificially by combining at least two polynucleotide components from different sources (natural or synthetic). For example, the constructs described herein comprise the coding region of a gene encoding the isolated BIN1 SH3 protein operably linked to a promoter. Constructs can be generated using conventional recombinant DNA methods. As used herein, the term “promoter” refers to a DNA sequence that regulates the transcription of a polynucleotide. Typically, a promoter is a regulatory region that is capable of binding RNA polymerase and initiating transcription of a downstream sequence. However, a promoter may be located at the 5′ or 3′ end, within a coding region, or within an intron of a gene that it regulates. Promoters may be derived in their entirety from a native gene, may be composed of elements derived from multiple regulatory sequences found in nature, or may comprise synthetic DNA segments. It is understood by those skilled in the art that different promoters may direct the expression of a gene in different tissues or cell types, at different stages of development, or in response to different environmental conditions. A promoter is “operably linked” to a polynucleotide if the promoter is connected to the polynucleotide such that it may affect transcription of the polynucleotide.

The polynucleotide may further comprise a sequence encoding a purification tag that allows purification or isolation of the BIN1 SH3 protein. Suitable tags are known in the art and include, but are not limited to, 6-Histidine (“6× His” or “His”), hemagglutinin (HA), cMyc, GST, Flag (DYKDDDDDK) tag, V5 tag, and NE-tag. A BIN SH3 domain protein comprising a His tag may comprise SEQ ID NO: 2 (HHHHHHSSGRENLYFQGHMGRLDLPPGFMFKVQAQHDYTATDTDELQLKAGDVVLVI PFQNPEEQDEGWLMGVKESDWNQHKELEKCRGVFPENFTERVP), or a sequence having at least 80%, 85%, 90%, 91%, 92%, 93%, 94%, 95%, 96%, 97%, 98%, or 99% identity thereto. The His-tagged isolated protein may consist of SEQ ID NO: 2.

In a third aspect, provided herein is an expression vector comprising any of the polynucleotide sequences or constructs encoding the BIN1 SH3 protein described herein.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. An “expression vector” or “recombinant expression vector” is a vector that is capable of directing the expression of exogenous genes. In general, expression vectors of utility in recombinant DNA techniques are often in the form of plasmids. In the present specification, “plasmid” and “vector” can be used interchangeably. However, the disclosed methods and compositions are intended to include such other forms of expression vectors, such as viral vectors (e.g., replication defective retroviruses, adenoviruses and adeno-associated viruses), and mammalian vectors which serve equivalent functions.

The expression vectors contemplated herein may add a number of amino acids to a protein encoded therein, such as to the amino terminus of the recombinant protein. Such vectors may serve one or more purposes, such as: (i) to increase expression of the recombinant protein; (ii) to increase the solubility of the recombinant protein; (iii) to aid in the purification of the recombinant protein by acting as a ligand in affinity purification (e.g., a His tag); (iv) to tag the recombinant protein for identification (e.g., such as Green fluorescence protein (GFP) or an antigen (e.g., an HA tag) that can be recognized by a labelled antibody); and (v) to promote localization of the recombinant protein to a specific area of the cell (e.g., a nuclear localization signal (NLS). Often, in expression vectors that add affinity purification tags to the recombinant protein include a proteolytic cleavage site at the junction of the tag and the recombinant protein to enable separation of the recombinant protein from the tag subsequent to purification of the recombinant protein. In exemplary embodiments, the expression vector adds a His tag to the BIN1 SH3 protein.

In a fourth aspect, provided herein is a host cell comprising any of the expression vectors described herein.

The vectors described herein may be introduced and propagated in a cell, which may be used to amplify copies of the vector and/or express the BIN1 SH3 protein. Suitable host cells include prokaryotic and eukaryotic cell. In preferred embodiments, the host cell is a bacterial cell. In exemplary embodiments, the host cell is E. coli. Incorporation of polynucleotides into a suitable expression vector for subsequent transformation of a host cell and subsequent selection of the transformed cells is well known to those skilled in the art as provided in Sambrook et al. (1989), Molecular cloning: A Laboratory Manual, Cold Spring Harbor Laboratory.

A variety of protein isolation and detection methods are known and can be used to isolate the BIN1 SH3 from the host cells described herein. Protein isolation and detection methods are well known in the art, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc .; Bollag et al. (1996) Protein Methods, 2nd Edition Wiley-Liss, NY; Walker (1996) The Protein Protocols Handbook Humana Press, NJ, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3rd Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ; and the references cited therein. Additional details regarding protein purification and detection methods can be found in Satinder Ahuja ed., Handbook of Bioseparations, Academic Press (2000).

In a fifth aspect, provided herein is a kit comprising the polynucleotide encoding the BIN SH3 protein described herein, and a cell-free protein synthesis system.

A “cell-free protein synthesis system,” “CFPS reaction mixture,” or “cell-free system” typically contains a crude or partially-purified cell extract and a suitable reaction buffer for promoting cell-free protein synthesis from a nucleic acid template. The nucleic acid template may include an RNA template and/or a DNA template. The DNA template may include an open reading frame operably linked to a promoter element for a DNA-dependent RNA polymerase. Additional NTP's and divalent cation cofactor may be included in the cell-free system. A reaction mixture is referred to as complete if it contains all reagents necessary to enable the reaction, and incomplete if it contains only a subset of the necessary reagents. It will be understood by one of ordinary skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of ordinary skill in the art that reaction components are packaged separately for commercialization and that useful commercial kits may contain any subset of the reaction components of the invention.

Cell-free systems may utilize components that are crude and/or that are at least partially isolated and/or purified. As used herein, the term “crude” may mean components obtained by disrupting and lysing cells and, at best, minimally purifying the crude components from the disrupted and lysed cells, for example by centrifuging the disrupted and lysed cells and collecting the crude components from the supernatant and/or pellet after centrifugation. The term “isolated or purified” refers to components that are removed from their natural environment, and are at least 60% free, preferably at least 75% free, and more preferably at least 90% free, even more preferably at least 95% free from other components with which they are naturally associated.

Cell-free protein synthesis (CFPS) is known and has been described in the art. (See, e.g., U.S. Pat. Nos. 6,548,276; 7,186,525; 8,734,856; 7,235,382; 7,273,615; 7,008,651; 6,994,986 7,312,049; 7,776,535; 7,817,794; 8,298,759; 8,715,958; 9,005,920; U.S. Publication No. 2014/0349353, U.S. Publication No. 2016/0060301, U.S. Publication No. 2018/0016612, and U.S. Publication No. 2018/0016614, the contents of which are incorporated herein by reference in their entireties).

Cell-free systems may comprise a cellular extract from a host strain. Because cell-free protein synthesis systems exploit an ensemble of catalytic proteins prepared from the crude lysate of cells, the cell extract (whose composition is sensitive to growth media, lysis method, and processing conditions) is an important component of extract-based CFPS reactions. A variety of methods exist for preparing an extract competent for cell-free protein synthesis, including those disclosed in U.S. Patent Application Publication No. 2014/0295492 and U.S. Patent Application Publication No. 2016/0060301, the contents of which are incorporated by reference in their entireties. The cellular extract of the platform may be prepared from a cell culture of a prokaryote (e.g., E. coli). While E. coli is exemplified herein, the bacterial species is not intended to be limiting. Other bacterial species suitable for the compositions and methods disclosed herein include but are not limited to (e.g., Bacillis species such as Bacillus subtilis, Vibrio species such as Vibrio natrigens, Pseudomonas species, etc.). In some embodiments, the cell culture is in stationary phase. In some embodiments, stationary phase may be defined as the cell culture having an OD600 of greater than about 3.0, 3.5, 4.0, 4.5, 5.0, 5.5, 6.0, 6.5, 7.0, 7.5, 8.0, or having an OD600 within a range bounded by any of these values. Further methods for preparing a cell-free system are disclosed in International Patent Application Publication No. WO2020185451A2, the contents of which are incorporated by reference in its entirety.

In a sixth aspect, provided herein is a method for identifying a candidate therapeutic for treating Alzheimer's disease by determining the binding affinity of an analyte to the BIN1 SH3 domain. A candidate therapeutic will have a sufficiently strong binding affinity to the domain. The method may comprise determining an equilibrium dissociation constant (K_{D}) between an analyte and an isolated protein, wherein the isolated protein comprises a sequence having at least 95% identity to SEQ ID NO: 1 (BIN1 SH3); wherein a K_{D} of less than about 15 μM identifies the analyte as a candidate therapeutic. In preferred embodiments, the K_{D} is between about 0.1 μM and about 15 μM, or any value or range in between. For example, the K_{D} may be about 0.1 μM, about 0.3 μM, about 0.5 μM, about 0.75 μM, about 10 μM, about 12.5 μM, or about 15 μM.

As used herein, the term “analyte” refers to any molecule capable of binding a protein, including a chemical compound, a protein, a nucleic acid, etc. In exemplary embodiments, the analyte is a chemical compound.

The term “Kassoc” or “Ka”, as used herein, refers to the association rate of a particular analyte-protein interaction, whereas the term “Kdis” or “Kd,” as used herein, refers to the dissociation rate of a particular antibody-antigen interaction. The term “K_{D}”, as used herein, refers to the dissociation constant, which is obtained from the ratio of K_{D} to Ka (i.e., K_{D}/Ka) and is expressed as a molar concentration (M). K_{D} values for antibodies can be determined using methods well established in the art. In exemplary embodiments, the method for determining the K_{D} of an analyte is by using surface plasmon resonance. Surface plasmon resonance may be done using a biosensor system such as a BIACORE® system. Other methods for determining the K_{D} may be used, such as by Bio-Layer Interferometry or flow cytometry.

The method may further comprise selecting an analyte using a molecular docking software before determining the K_{D} with BIN1 SH3). From high ranking analytes on the software (those having the best molecular docking scores), molecules with optimal complementarity with the protein binding pocket may be selected for experimental testing.

### Miscellaneous

Unless otherwise specified or indicated by context, the terms “a”, “an”, and “the” mean “one or more.”

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

### EXAMPLES

The following Examples are illustrative and should not be interpreted to limit the scope of the claimed subject matter.

### Example 1

Genome-wide association studies have identified the human Bridging INtegrator 1 (BIN1) gene within the second most common susceptibility locus for late-onset Alzheimer's disease (LOAD) [1]. Alternate splicing of BIN1 generates more than 15 tissue- and cell-type-specific and ubiquitous isoforms that participate in multiple cellular functions, including endocytosis, membrane remodeling, actin cytoskeleton regulation, DNA repair, and apoptosis [2, 3]. In humans and mice, the loss, downregulation, or aberrant alternate splicing of BIN1 in peripheral tissue has been linked to centronuclear myopathy, myotonic dystrophy, ventricular cardiomyopathy, ventricular arrhythmia, and cancer progression [2]. Most LOAD-associated BIN1 single-nucleotide polymorphisms and an indel lie several kb upstream of the BIN1 coding region and have been proposed to increase the risk for Alzheimer's disease by altering cellular BIN1 expression [4-6].

In the brain, BIN1 is expressed in neurons, oligodendrocytes, microglia, and at lower levels in astrocytes [3, 7, 8]. In neurons, BIN1 localizes to pre- and post-synaptic sites. [3, 9-11]. The inventors' recent characterization of hippocampal CA1 excitatory synapses by slice electrophysiology revealed BIN1's indispensable role in synaptic transmission by regulating neurotransmitter vesicle dynamics at the presynaptic terminal [12]. In agreement, neuron-specific BIN1 expression has been correlated with excitability in cultured primary neurons.[13, 14]. Thus, neuronal BIN1 has a fundamental function in synaptic physiology. Relatively little is known about BIN1's function in oligodendrocytes. Recently, the inventors reported that microglial BIN1 plays an essential role in neuroinflammation by regulating the activation of proinflammatory gene expression.[8]

Interestingly, the BIN1 insertion risk allele s59335482 was associated with higher post-mortem tau pathology but not Aβ when compared to non-carrier Alzheimer's disease patients [4]. Furthermore, among older individuals without dementia, carriers of the BIN1 rs744373 risk-allele were found to have similar amyloid pathology but increased tau pathology and significantly impaired memory performance [15]. Independent groups have reported a significant decrease in neuronal BIN1 expression in the brains of patients with LOAD [3, 16-20]. Neuronal loss in BIN1 neuronal cKO mice was observed in the context of tauopathy, but how BIN1 loss modified tau pathogenesis was not characterized [13]. A more recent study reported the identification of BIN1 rs6733839-T as a risk allele associated with Lewy body dementia [21].

It is widely accepted that tau pathology propagates via cell-to-cell spread across neuronal circuits [22, 23]. BIN1's role in endocytosis and synaptic activity could profoundly influence tau pathology propagation. However, there is inconsistency in vitro data regarding BIN1's role in tau pathology. In one study, the loss of BIN1 in cultured neurons was found to facilitate neuron-to-neuron tau pathology propagation by promoting aggregate endocytosis [24]. However, another study found that BIN1 knockdown increases phosphorylated tau (p-tau) levels in synapses but reduces basal and stimulated tau release in cultured neurons [25]. Existing evidence suggests at least three different modes by which BIN1 might impact tau pathology. First, cytosolic BIN1 limits extracellular pathogenic tau seed uptake and neuron-to-neuron propagation in primary cultures [24]. Second, the BIN1 SH3 domain interacts with the proline-rich motif in tau in the cytosol, and tau phosphorylation at Thr231 weakens this interaction. This interaction was speculated to influence Alzheimer's disease risk by an unknown mechanism [4, 26]. Third, microglial BIN1 was found to influence the release of tau in extracellular vesicles [27].

In order to elucidate how BIN1 function relates to disease risk for Alzheimer's disease, it is imperative to understand better BIN1's role in tau pathogenesis and disease progression using appropriate animal models. The inventors recently generated mice lacking BIN1 expression in forebrain neurons and oligodendrocytes (Bin1-cKO) in the P301S tau transgenic background (line PS19) [28] and analyzed how BIN1 influences tau pathophysiology. The results showed that BIN1 influences tau pathogenesis in a region-specific manner. Whereas the loss of BIN1 exacerbated tau inclusions in the somatosensory cortex and spinal cord, it attenuated pathology in the hippocampus, entorhinal/piriform cortex, and amygdala. Notably, BIN1 loss preserved hippocampal synapses, decreased neuroinflammation, and resulted in complex gene expression changes that underlie cell-autonomous and non-cell-autonomous responses. Collectively, these in vivo results demonstrated that BIN1 promotes region-specific tauopathy and neuroinflammation, which is highly relevant to Alzheimer's disease pathophysiology.

The above findings revealed that BIN1 promotes tau pathogenesis. Thus, the inventors pursued investigations to develop small-molecule inhibitors for BIN1. As mentioned above, BIN1 is expressed as multiple isoforms in the brain and peripheral tissue. All BIN1 isoforms contain the N-terminal BAR domain and the C-terminal SH3 domain. The PI, CLAP, and MBP domains are variably spliced in cell- and tissue-specific BIN1 isoforms.

As the first step in the drug discovery process, the inventors solved the crystal structure of the BIN1 SH3 domain, which is present in all BIN1 isoforms.

### Methods

Protein Expression & Purification: A cDNA encoding 6× His epitope tagged human BIN1 SH3 domain was inserted into pET28a (+) bacterial expression vector with NdeI/XhoI digestion sites. The protein sequence for the N-terminally His-tagged BIN1-SH3 is

(SEQ ID NO: 2)
HHHHHHSSGRENLYFQGHMGRLDLPPGFMFKVQAQHDYTATDTDELQLKA
GDVVLVIPFQNPEEQDEGWLMGVKESDWNQHKELEKCRGVFPENFTERV
P.

For 6× HIS-SH3 expression and purification, the plasmid was transformed into BL21 (DE3) cells. A single colony was picked and inoculated in 50 mL LB broth with kanamycin (50 μg/mL) overnight. 10 mL of overnight culture was used to inoculate 1 L of LB broth with 1 mL kanamycin (50 mg/mL). The 1 L culture was grown at 250 rpm at 37° C. until optical density (OD) reached 0.6-0.8. Expression was induced with 0.5 mM isopropyl-β-d-thiogalactopyranoside (IPTG) at 20° C. overnight, then pelleted at 5k×g for 10 min and resuspended in 10 mL lysis buffer [20 mM tris (pH 8.4), 300 mM NaCl, 10% glycerol v/v, and 20-mM imidazole], containing protease inhibitors. Cells were lysed by sonication on a 10 s sonication/15 s rest cycle for 15 min at an amplitude of 6. The lysate was centrifuged at 40k×g for 40 min at 4° C. and the supernatant was filtered, then loaded onto a HisTrap HP column at 1 mL/min after equilibration with lysis buffer, 5% His-prep buffer [20 mM Tris (pH 8.4), 300 mM NaCl, 10% Glycerol v/v, 500 mM imidazole], then again in lysis buffer. The protein was eluted by linear gradient (0%-100%) of His-prep buffer at 1 mL/min and the peak of the protein was pooled, concentrated, and diluted to 30 mL in TEV cleavage buffer [20 mM Tris (pH 8.0), 100 mM NaCl, and 10% glycerol v/v]. For cleavage, 2 mg of the protease TEV was added and incubated at 20° C. overnight. His-prep buffer was added to the overnight sample at a final concentration of 5%. The HisTrap HP column was washed with His-prep buffer, then 5% His-prep buffer before loading the cleaved sample at 1 mL/min. The flowthrough containing the untagged BIN1-SH3 was collected and concentrated, then loaded on the HiPrep 16/60 Sephacryl S-200 HR gel filtration column. The peak fractions were pooled and concentrated to 23.1 mg/ml (1 OD=0.77 mg/mL), then stored at −80° C. Protein purity was evaluated by SDS-polyacrylamide gel electrophoresis (PAGE).

The protein sequence for the untagged BIN1-SH3 domain is

(SEQ ID NO: 1)
GRLDLPPGFMFKVQAQHDYTATDTDELQLKAGDVVLVIPFQNPEEQDEGW
LMGVKESDWNQHKELEKCRGVFPENFTERVP.

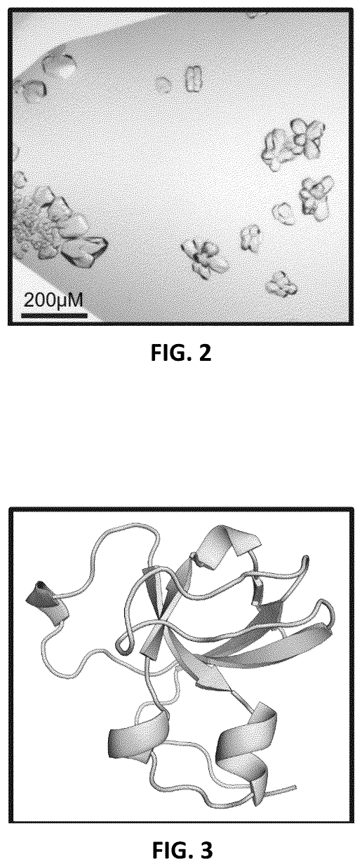

Protein Crystallization, Data Collection, and Refinement: Crystals were grown using purified human BIN1-SH3 at 23.1 mg/mL mixed with an equal volume of crystallization buffer (1:1) in a vapor diffusion, hanging drop apparatus at 20° C. Crystals grew within 1-2 days (FIG. 2). Crystals were harvested and then flash-frozen in liquid nitrogen for X-ray diffraction. A separate cryoprotectant buffer for freezing was not needed, as the crystallization buffer had cryoprotectant properties.

X-ray diffraction data for the human BIN1-SH3 domain structure was collected on the Structural Biology Center (SBC) 19-BM beamline at the Advanced Photon Source (APS) in Argonne National Laboratory, IL on Oct. 29, 2020, and data was processed using HKL3000. BALBES was used for molecular replacement, and the structure of PDB 1BB9 was used as the template. The CCP4 suite and Coot were used to complete the model building and refinement. PyMOL was used to generate figures. The structure was solved at 1.54 Å in the P2₁ space group with four copies of the protein in the asymmetric unit (FIG. 3).

### Example 2

After determining the crystal structure of the human BIN1 SH3 domain, binding of compounds to this peptide was assessed.

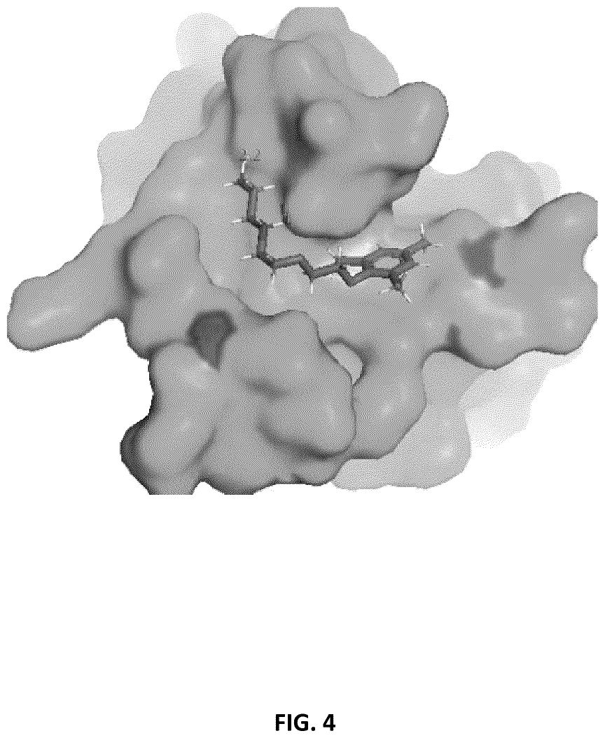

Molecular docking software (DOCK 3.6) was used in combination with the ZINC20 drug database to virtually screen compounds. FIG. 4 illustrates docking of a lead-like compound to the peptide.

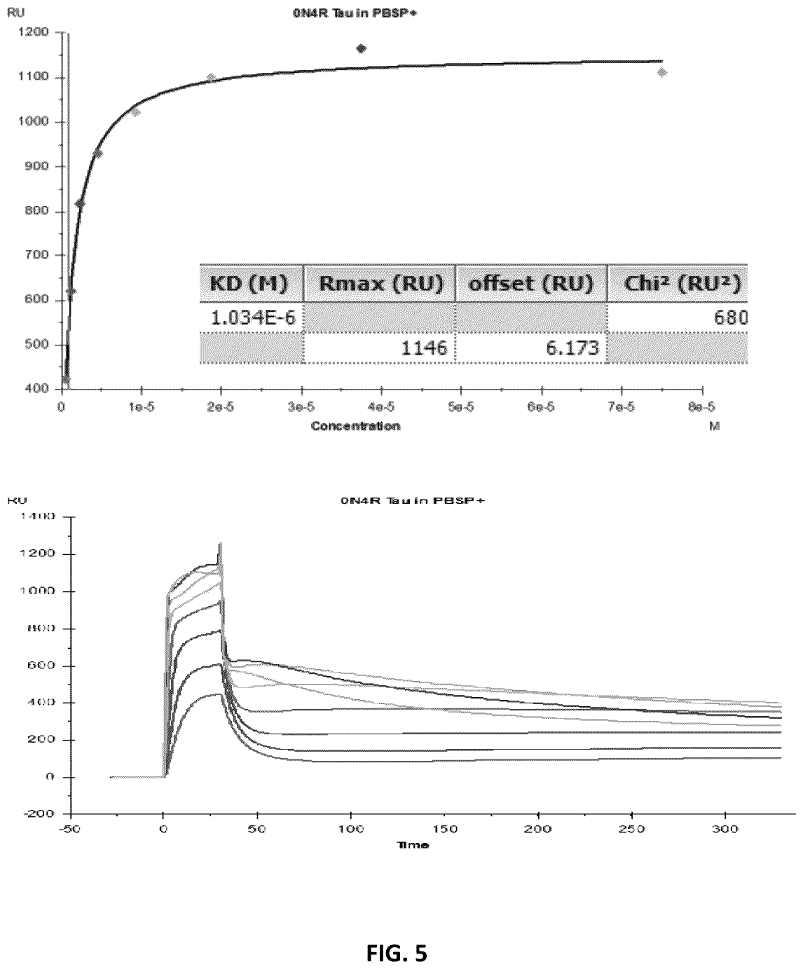

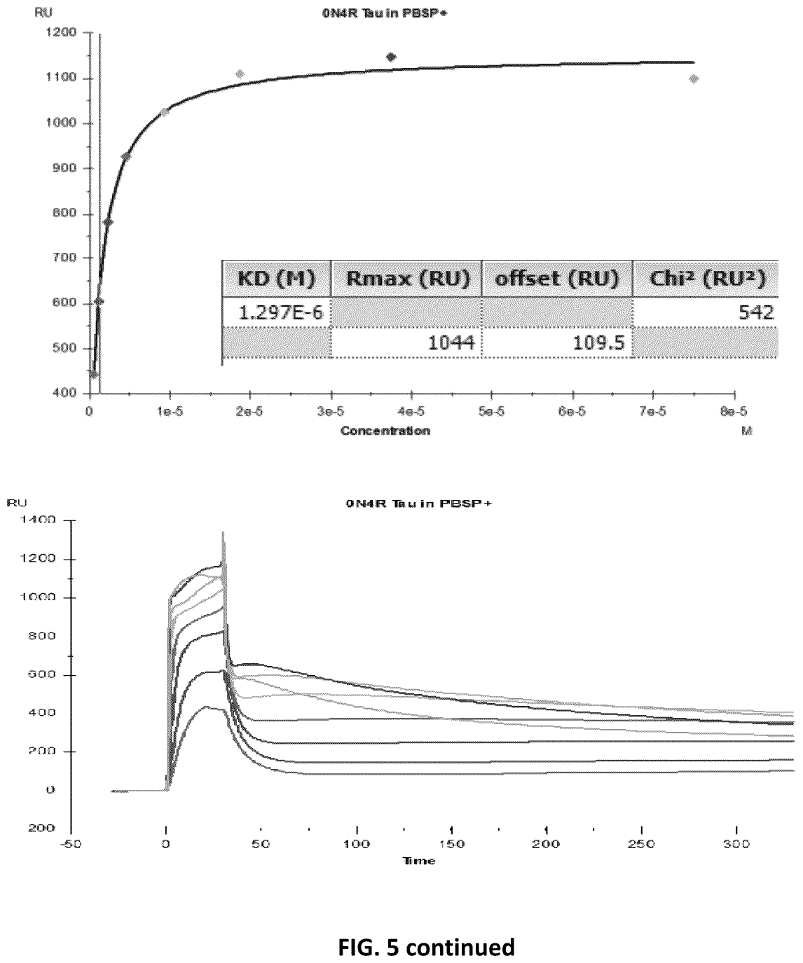

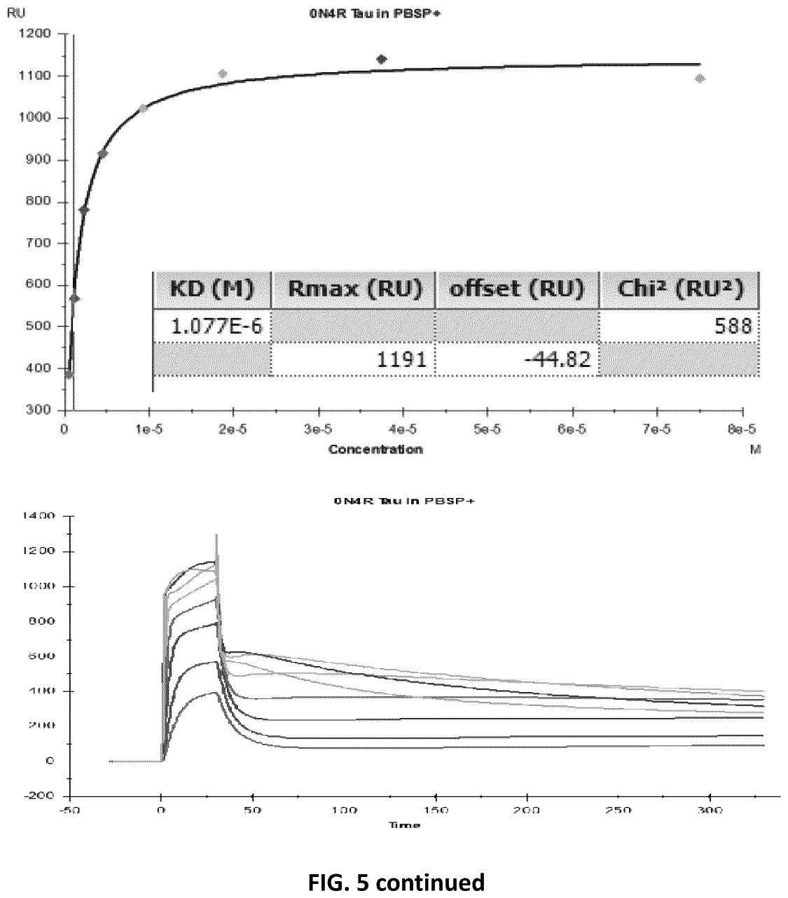

Next, the inventors determined the dissociation constant (K_{D}) for the peptide and full-length 0N4R WT Tau by surface plasmon resonance (SPR). The experiment was run in triplicate with BIN1 SH3 domain 50 μg/mL as the ligand, and FL 0N4R WT Tau in serial dilution from 75-0.6 μM as the analyte. Flow rate—30 μL/minute, contact time—30 seconds, dissociation time—120 seconds, temperature—25° C., running buffer—1×PBS-P+. The results are shown in FIG. 5. A dissociation constant of 1.136 μM was determined.

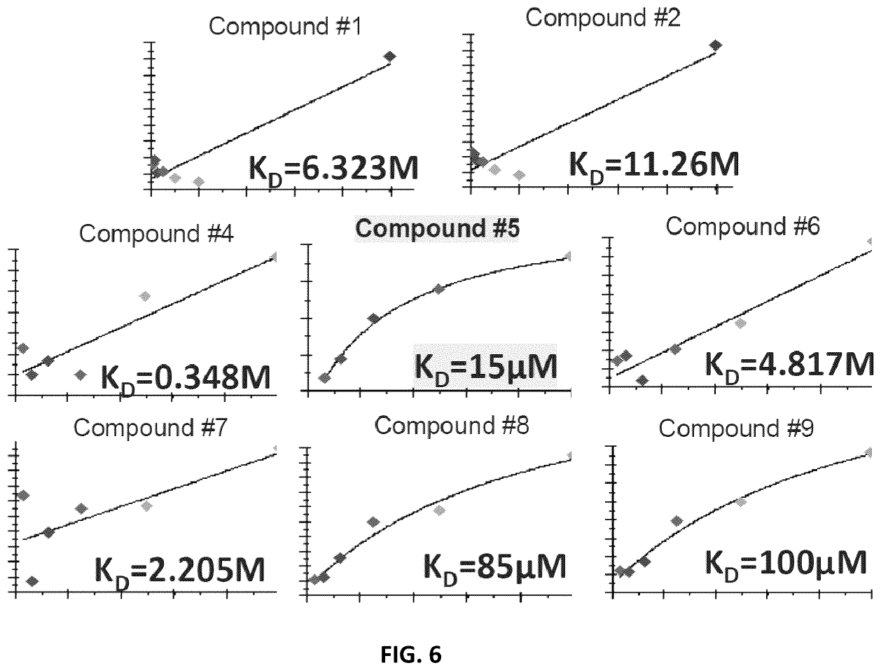

Then, the dissociation constants for nine compounds identified through the molecular docking analysis were assessed. The results are shown in FIG. 6. Compound #5 was shown to have a similar dissociation constant as that of FL 0N4R WT Tau (K_{D}=15 μM). Therefore, compound #5 is a candidate compound for further testing in Alzheimer's disease. Ligand-BIN1 SH3 domain 50 μg/mL. Analyte—compounds 1-2 (serial dilutions from 500-3.12 μM), #4 & 6-9 (250-3.12 μM) and #5 (dilutions from 100-3.12 μM). Flow rate—30 μL/minute, contact time—30 seconds, dissociation time—300 seconds, temperature—25° C., running buffer—1.05×PBS-P+ with 5% DMSO. Solvent correction for 5% DMSO was calculated and mathematically subtracted from each compound analysis.

### REFERENCES

1. Karch C M, Goate A M: Alzheimer's disease risk genes and mechanisms of disease pathogenesis. Biol Psychiatry 2015, 77:43-51.
2. Prokic I, Cowling B S, Laporte J: Amphiphysin 2 (BIN1) in physiology and diseases. J Mol Med (Berl) 2014, 92:453-463.
3. De Rossi P, Buggia-Prevot V, Clayton B L, Vasquez J B, van Sanford C, Andrew R J, Lesnick R, Botte A, Deyts C, Salem S, et al: Predominant expression of Alzheimer's disease-associated BIN1 in mature oligodendrocytes and localization to white matter tracts. Mol Neurodegener 2016, 11:59.
4. Chapuis J, Hansmannel F, Gistelinck M, Mounier A, Van Cauwenberghe C, Kolen K V, Geller F, Sottejeau Y, Harold D, Dourlen P, et al: Increased expression of BIN1 mediates Alzheimer genetic risk by modulating tau pathology. Mol Psychiatry 2013, 18:1225-1234.
5. Martiskainen H, Viswanathan J, Nykänen N P, Kurki M, Helisalmi S, Natunen T, Sarajärvi T, Kurkinen K M, Pursiheimo J P, Rauramaa T, et al: Transcriptomics and mechanistic elucidation of Alzheimer's disease risk genes in the brain and in vitro models. Neurobiol Aging 2015, 36:1221.e1215-1228.
6. Nott A, Holtman I R, Coufal N G, Schlachetzki J C M, Yu M, Hu R, Han C Z, Pena M, Xiao J, Wu Y, et al: Brain cell type-specific enhancer-promoter interactome maps and disease-risk association. Science 2019, 366:1134-1139.
7. Adams S L, Tilton K, Kozubek J A, Seshadri S, Delalle I: Subcellular Changes in Bridging Integrator 1 Protein Expression in the Cerebral Cortex During the Progression of Alzheimer Disease Pathology. J Neuropathol Exp Neurol 2016, 75:779-790.
8. Sudwarts A, Ramesha S, Gao T, Ponnusamy M, Wang S, Hansen M, Kozlova A, Bitarafan S, Kumar P, Beaulieu-Abdelahad D, et al: BIN1 is a key regulator of proinflammatory and neurodegeneration-related activation in microglia. Mol Neurodegener 2022, 17:33.
9. De Rossi P, Buggia-Prevot V, Andrew R J, Krause S V, Woo E, Nelson P T, Pytel P, Thinakaran G: BIN1 localization is distinct from Tau tangles in Alzheimer's disease. Matters (Zur) 2017, 2017.
10. Schurmann B, Bermingham D P, Kopeikina K J, Myczek K, Yoon S, Horan K E, Kelly C J, Martin-de-Saavedra M D, Forrest M P, Fawcett-Patel J M, et al: A novel role for the late-onset Alzheimer's disease (LOAD)-associated protein Bin1 in regulating postsynaptic trafficking and glutamatergic signaling. Mol Psychiatry 2020, 25:2000-2016.
11. Ramjaun A R, Micheva K D, Bouchelet I, McPherson P S: Identification and characterization of a nerve terminal-enriched amphiphysin isoform. J Biol Chem 1997, 272:16700-16706.
12. De Rossi P, Nomura T, Andrew R J, Masse N Y, Sampathkumar V, Musial T F, Sudwarts A, Recupero A J, Le Metayer T, Hansen M T, et al: Neuronal BIN1 Regulates Presynaptic Neurotransmitter Release and Memory Consolidation. Cell Rep 2020, 30:3520-3535.e3527.
13. McAvoy K M, Rajamohamed Sait H, Marsh G, Peterson M, Reynolds T L, Gagnon J, Geisler S, Leach P, Roberts C, Cahir-McFarland E, et al: Cell-autonomous and non-cell autonomous effects of neuronal BIN1 loss in vivo. PLOS One 2019, 14:e0220125.
14. Voskobiynyk Y, Roth J R, Cochran J N, Rush T, Carullo N V, Mesina J S, Waqas M, Vollmer R M, Day J J, McMahon L L, Roberson E D: Alzheimer's disease risk gene BIN1 induces Tau-dependent network hyperexcitability. Elife 2020, 9.
15. Franzmeier N, Neitzel J, Rubinski A, Smith R, Strandberg O, Ossenkoppele R, Hansson O, Ewers M: Functional brain architecture is associated with the rate of tau accumulation in Alzheimer's disease. Nat Commun 2020, 11:347.
16. Glennon E B, Whitehouse I J, Miners J S, Kehoe P G, Love S, Kellett K A, Hooper N M: BIN1 is decreased in sporadic but not familial Alzheimer's disease or in aging. PLOS One 2013, 8:e78806.
17. Holler C J, Davis P R, Beckett T L, Platt T L, Webb R L, Head E, Murphy M P: Bridging integrator 1 (BIN1) protein expression increases in the Alzheimer's disease brain and correlates with neurofibrillary tangle pathology. J Alzheimers Dis 2014, 42:1221-1227.
18. Yu L, Chibnik L B, Srivastava G P, Pochet N, Yang J, Xu J, Kozubek J, Obholzer N, Leurgans S E, Schneider J A, et al: Association of Brain DNA methylation in SORL1, ABCA7, HLA-DRB5, SLC24A4, and BIN1 with pathological diagnosis of Alzheimer disease. JAMA Neurol 2015, 72:15-24.
19. McKenzie A T, Moyon S, Wang M, Katsyv I, Song W M, Zhou X, Dammer E B, Duong D M, Aaker J, Zhao Y, et al: Multiscale network modeling of oligodendrocytes reveals molecular components of myelin dysregulation in Alzheimer's disease. Mol Neurodegener 2017, 12:82.
20. Marques-Coelho D, Iohan L, Melo de Farias A R, Flaig A, Lambert J C, Costa M R: Differential transcript usage unravels gene expression alterations in Alzheimer's disease human brains. NPJ Aging Mech Dis 2021, 7:2.
21. Chia R, Sabir M S, Bandres-Ciga S, Saez-Atienzar S, Reynolds R H, Gustavsson E, Walton R L, Ahmed S, Viollet C, Ding J, et al: Genome sequencing analysis identifies new loci associated with Lewy body dementia and provides insights into its genetic architecture. Nat Genet 2021, 53:294-303.
22. Frost B, Diamond M I: Prion-like mechanisms in neurodegenerative diseases. Nat Rev Neurosci 2010, 11:155-159.
23. Dujardin S, Hyman B T: Tau Prion-Like Propagation: State of the Art and Current Challenges. Adv Exp Med Biol 2019, 1184:305-325.
24. Calafate S, Flavin W, Verstreken P, Moechars D: Loss of Bin1 Promotes the Propagation of Tau Pathology. Cell Rep 2016, 17:931-940.
25. Glennon E B, Lau D H, Gabriele R M C, Taylor M F, Troakes C, Opie-Martin S, Elliott C, Killick R, Hanger D P, Perez-Nievas B G, Noble W: Bridging Integrator-1 protein loss in Alzheimer's disease promotes synaptic tau accumulation and disrupts tau release. Brain Commun 2020, 2.
26. Sottejeau Y, Bretteville A, Cantrelle F X, Malmanche N, Demiaute F, Mendes T, Delay C, Alves Dos Alves H, Flaig A, Davies P, et al: Tau phosphorylation regulates the interaction between BIN1's SH3 domain and Tau's proline-rich domain. Acta Neuropathol Commun 2015, 3:58.
27. Crotti A, Sait H R, McAvoy K M, Estrada K, Ergun A, Szak S, Marsh G, Jandreski L, Peterson M, Reynolds T L, et al: BIN1 favors the spreading of Tau via extracellular vesicles. Sci Rep 2019, 9:9477.
28. Yoshiyama Y, Higuchi M, Zhang B, Huang S M, Iwata N, Saido T C, Maeda J, Suhara T, Trojanowski J Q, Lee V M: Synapse loss and microglial activation precede tangles in a P301S tauopathy mouse model. Neuron 2007, 53:337-351.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

We claim:

1. An isolated protein comprising a sequence having at least 95% identity to SEQ ID NO: 1

2. The isolated protein of claim 1, wherein the sequence comprises SEQ ID NO: 1.

3. The isolated protein of claim 1, wherein the sequence consists of SEQ ID NO: 1.

4. The isolated protein of claim 1, wherein the protein is linked to a solid support.

5. A polynucleotide comprising a sequence encoding the isolated protein of claim 1.

6. The polynucleotide of claim 5, further comprising a sequence encoding a purification tag.

7. The polynucleotide of claim 6, wherein the purification tag is a His tag.

8. The polynucleotide of claim 7, wherein a sequence encoding the isolated protein and the His tag has at least 95% identity to SEQ ID NO: 2.

9. An expression vector comprising the polynucleotide of claim 5.

10. The expression vector of claim 9, wherein the expression vector is a bacterial vector.

11. A host cell comprising the expression vector of claim 9.

12. The host cell of claim 11, wherein the host cell is a bacterial cell.

13. A kit comprising the polynucleotide of claim 5, and a cell-free protein synthesis system.

14. A method for identifying a candidate therapeutic for treating Alzheimer's disease, the method comprising:
determining an equilibrium dissociation constant (K_{D}) between an analyte and an isolated protein, wherein the isolated protein comprises a sequence having at least 95% identity to SEQ ID NO: 1;
wherein a K_{D} of less than about 15 μM identifies the analyte as a candidate therapeutic.

15. The method of claim 14, wherein a K_{D} of between about 0.1 μM and about 15 μM identifies the analyte as a candidate therapeutic.

16. The method of claim 14, wherein the step of determining the K_{D} is done by surface plasmon resonance.

17. The method of claim 14, wherein the analyte is a chemical compound.


SEQUENCE LISTING (2 sequences)

SEQ ID NO: 1 - protein, 81 aa, synthetic construct
GRLDLPPGFMFKVQAQHDYTATDTDELQLKAGDVVLVIPFQNPEEQDEGWLMGVKESDWNQHKELEKCRGVFPENFTERVP

SEQ ID NO: 2 - protein, 100 aa, synthetic construct
HHHHHHSSGRENLYFQGHMGRLDLPPGFMFKVQAQHDYTATDTDELQLKAGDVVLVIPFQNPEEQDEGWLMGVKESDWNQHKELEKCRGVFPENFTERVP